(19) United States
(12) Patent Application Publication
(10) Pub. No.: US 2023/0389835 A1
(43) Pub. Date: Dec. 7, 2023

(54) APPARATUS AND METHOD FOR DETERMINING HEMOGLOBIN LEVELS

(71) Applicant: MY-OR DIAGNOSTICS LTD., Zikhron Yaakov (IL)

(72) Inventors: Michal ASHKENAZI, Zurit (IL); Anna PASECHNEK, Tel Aviv (IL); Inbal ESHET-KESSLER, Koranit (IL); Idan KATZ, Zikhron Yaakov (IL); Ron KOHN, Kefar Uriya (IL); Ariel KATZ, Zikhron Yaakov (IL); Michael BRANDWEIN, Elazar (IL)

(21) Appl. No.: 18/022,667

(22) PCT Filed: Aug. 23, 2021
(86) PCT No.: PCT/IL2021/051031

§ 371 Date: Feb. 22, 2023

Related U.S. Application Data
(60) Provisional application No. 63/069,069, filed on Aug. 23, 2020.

(51) Int. Cl. A61B 5/1455; A61B 5/00; A61B 5/145

(52) U.S. Cl. CPC A61B 5/1455; A61B 5/14546; A61B 5/0077

(57) ABSTRACT

An apparatus, a kit and a method for non-invasively determining hemoglobin levels are provided. The apparatus comprising: a box defining an interior space comprising: (a) un upper opening adopted to allow a camera placed on top of the box, to capture images of a subject's hand placed inside the interior space; and (b) a side opening configured for an insertion of said subject's hand; an adaptor, attached to an upper face to the box, for holding the camera and ensuing a location of the camera lens above the upper opening; a placement pad located at a bottom of the box, having a texture configured to direct said subject to place a hand portion comprising a nail and skin in a direction facing a lens of said camera, such that, a field of view of said camera captures the nail and the skin.

### CROSS REFERENCE TO RELATED APPLICATIONS

This application claims the benefit of priority to U.S. Provisional Patent Application No. 63/069,069 filed Aug. 23, 2020, entitled “APPARATUS AND METHOD FOR DETERMINING HEMOGLOBIN LEVELS”, the contents of which are incorporated herein by reference in their entirety.

### FIELD OF THE INVENTION

Applications of the present invention relate generally to an apparatus and a method for non-invasively determining hemoglobin levels.

### BACKGROUND

Hemoglobin is an iron-rich protein that helps red blood cells carry oxygen from the lungs to the rest of the body. The iron in hemoglobin carries oxygen to fuel the many processes of metabolism that occur throughout our bodies, and it is necessary to maintain healthy cells, skin, hair, and nails.

Low levels of hemoglobin in red blood cells is marked by iron deficiency, which is the most common micronutrient deficiency worldwide and the leading cause of anemia in the United States. Iron deficiency is due either to increased need for iron by the body or a decreased absorption or amount of iron taken in. Signs of iron deficiency can include fatigue, decreased work and school performance, difficulty maintaining body temperature, decreased immune function, and glossitis.

Young children are a special risk group for iron deficiency because their rapid growth leads to high iron requirements. Infants and children who are deficient in iron may experience delayed growth and development, including delayed neurodevelopment, and may be prone to infection.

Existing approaches for diagnostic of hemoglobin levels require invasive blood test, accordingly, there is a need for new apparatuses and method for diagnostic of hemoglobin levels by using non-invasive and more accessible approaches.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

Some aspects of the invention are directed to an apparatus for determining hemoglobin levels in a subject, said apparatus comprising:
(i) a box defining an interior space comprising:
(a) un upper opening adopted to allow a camera placed on top of the box, to capture images of a subject's hand placed inside the interior space, wherein a vertical distance between the upper opening and the bottom of the box is between 50 to 400 mm; and
(b) a side opening configured for an insertion of said subject's hand;
(ii) an adaptor, attached to an upper face to the box, for holding the camera and ensuing a location of the camera lens above the upper opening;
(iii) a placement pad located at a bottom of the box, having a texture configured to direct said subject to place a hand portion comprising a nail and skin in a direction facing a lens of said camera; and
wherein a field of view of said camera captures the nail and the skin.

In some embodiments, the apparatus further comprises a color scale bar, located at said bottom of the box, displaying at least 3 colors characterized by a reflection of wavelength in the range of (i) 670-700 nm; (ii) 520-560 nm; and (iii) 450-490 nm, wherein a field of view of said camera also captures the color scale bar.

In some embodiments, the apparatus further comprises a cover connected to the box, for covering the side opening for substantially blocking an amount of exterior light to be inserted within said interior space upon applying the camera on the adaptor and the subject's hand within said opening. In some embodiments, the maximum amount of exterior light to be captured by the camera is at most 1 Luman.

In some embodiments, the camera is included in a smartphone.

In some embodiments, the interior space has dimensions sufficient for an insertion of the hand of the subject at a spread-out position. In some embodiments, the color scale bar further displays at least one grayscale portion.

Some additional aspects of the invention are directed to a kit comprising:
i. a box defining an interior space comprising:
(a) un upper opening adopted to allow a camera placed on top of the box to capture images of a subject's hand placed inside the interior space, wherein a vertical distance between the upper opening and the bottom of the box is between 50 to 400 mm; and
(b) a side opening configured for insertion of at least a subject's hand;
ii. an adaptor, attachable to an upper face of the box, for holding the camera and ensuing a location of the camera lens above the upper opening; and
iii. a placement pad to be located at a bottom of the box, having a texture configured to direct said subject to place a hand portion comprising a nail and skin in a direction facing a lens of said camera;
wherein a field of view of said camera captures the nail and the skin.

In some embodiments, the kit further comprises a color scale bar, adopted to be located at said bottom of the box, displaying at least 3 colors characterized by a reflection of wavelength in the range of (i) 670-700 nm; (ii) 520-560 nm; and (iii) 450-490 nm, wherein a field of view of said camera also captures the color scale bar.

In some embodiments, the kit further comprises a cover, connectable to the box, and adopted to a cover the side opening for substantially blocking an amount of exterior light to be inserted within said interior space upon applying the camera on the adaptor and the subject's hand within said opening.

Some additional aspects of the invention are directed to a method for determining hemoglobin levels in a subject in need thereof using the apparatus according to any of the embodiments disclosed herein, the method operating to execute program instructions, and comprising the following steps:
i. receiving an image from said camera captured under a defined light condition, the image comprising a plurality of nails and skin of the subject and the color scale bar;
ii. performing an image segmentation of the pixels of the nail portions of the image;
iii. transforming the image data into hemoglobin levels by trained neural network; and
iv. outputting for display the hemoglobin level of the subject.

In some embodiments, the program instructions further receive as input a video from the camera.

In some embodiments, the program instructions further analyze a heart rate of the subject from said video.

In some embodiments, the program instructions further receive as input one or more parameters of the subject selected from of the group consisting of: age, gender, and heart rate.

Some additional aspects of the invention are directed to a method of non-invasive rapid blood hemoglobin levels measurement with a mean absolute error of less that 0.95 g/dL, the method comprising receiving an image of nails of a subject captured from a camera under a defined light condition of at most 1 Luman. In some embodiments, the method further includes placing a portion of a hand of a user comprising a nail and a skin in an apparatus according to any one of the embodiments disclosed herein.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1A presents a perspective view depicting a non-limiting exemplary configuration of an apparatus for determining hemoglobin levels according to some embodiments of the invention.

FIG. 1B presents a perspective view depicting another non-limiting exemplary configuration of an apparatus for determining hemoglobin levels according to some embodiments of the invention.

FIG. 1C presents another perspective view depicting another non-limiting exemplary configuration of the apparatus of FIG. 1B.

FIG. 2A presents a top view depicting a non-limiting exemplary configuration of the inner portion of the apparatus, wherein the field of view of the camera captures both the back of the subject's hand, as well as the color scale bar according to some embodiments of the invention.

FIG. 2B presents a top view depicting another non-limiting exemplary configuration of the inner portion of the apparatus, wherein the field of view of the camera captures both the back of the subject's hand, as well as the color scale bar according to some embodiments of the invention.

FIG. 3 presents a flowchart of a non-limiting method for determining hemoglobin levels in a subject in need thereof according to some embodiments of the invention.

### DETAILED DESCRIPTION

### Apparatus

The invention provides, in some embodiments, an apparatus for determining hemoglobin levels in a subject in need thereof. None limiting example for use of the apparatus provided herein includes non-invasive detection, prevention and treatment of anemia as well as providing personal care of a subject afflicted by or suspected of having anemia.

In some embodiments, the apparatus comprises (i) a box defining an interior space comprising: un upper opening adopted to allow a camera, (e.g., with a flash device) placed on top of the box, to capture images of a subject's hand placed inside the interior space, wherein a vertical distance between the upper opening and the bottom of the box is between 50 to 400 mm, a side opening configured for an insertion of said subject's hand (e.g., an infant hand), (ii) an adaptor, attached to an upper face to the box, for holding (e.g., mounting) the camera, and (iii) a placement pad located at a bottom of the box, having a texture configured to direct said subject to place a hand portion comprising a nail and skin in a direction facing a lens of said camera. In some embodiments, the camera is mounted such that a field of view of said camera captures the nail and the skin.

In some embodiments, the device may further include (iv) a color scale bar positioned in the field of view of the camera such as to assist in an image segmentation process of the nail portion and optionally skin of the image captured by the camera. Further, the invention provides a method for determining hemoglobin levels by analyzing a photo of the fingernails of a subject.

The apparatus, in some embodiments thereof, is in a form of a closed box having an interior space, defined by walls. The apparatus may be made of rigid or elastic materials, having a rigid shell. The apparatus may have any geometric configuration such as circular, square, or elliptical, as long as the field of view of the camera includes both the nail portion of the user and the color scale bar. In some embodiments, the box is square and may have a length in the range of 60-140 mm, a height in the range of 8-16 cm, and a depth in the range of 40-120 mm. In some embodiments, the distance between the camera and the subject's hand may be in the range between 60-220 mm. In a nonlimiting example the box can be a recycled box of infants' milk substitute.

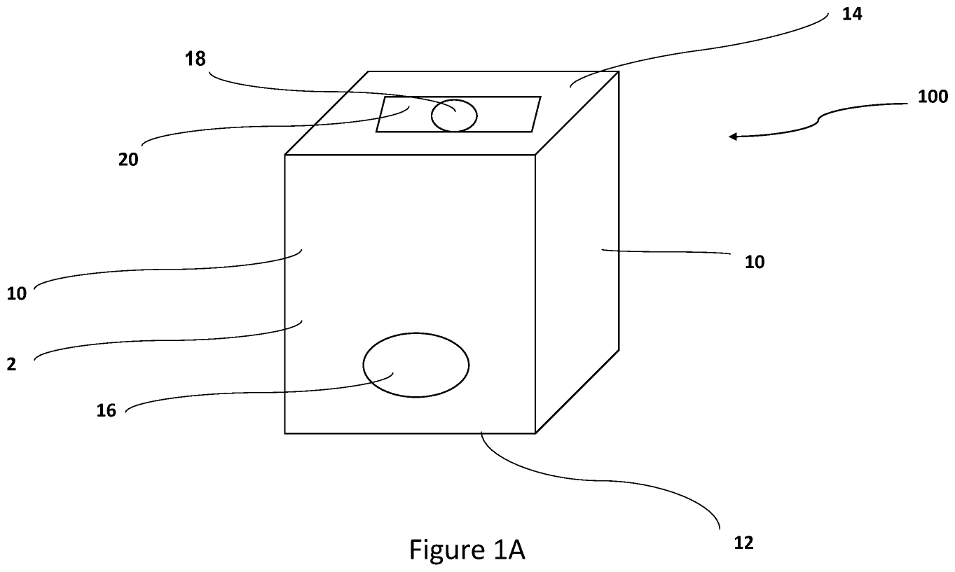

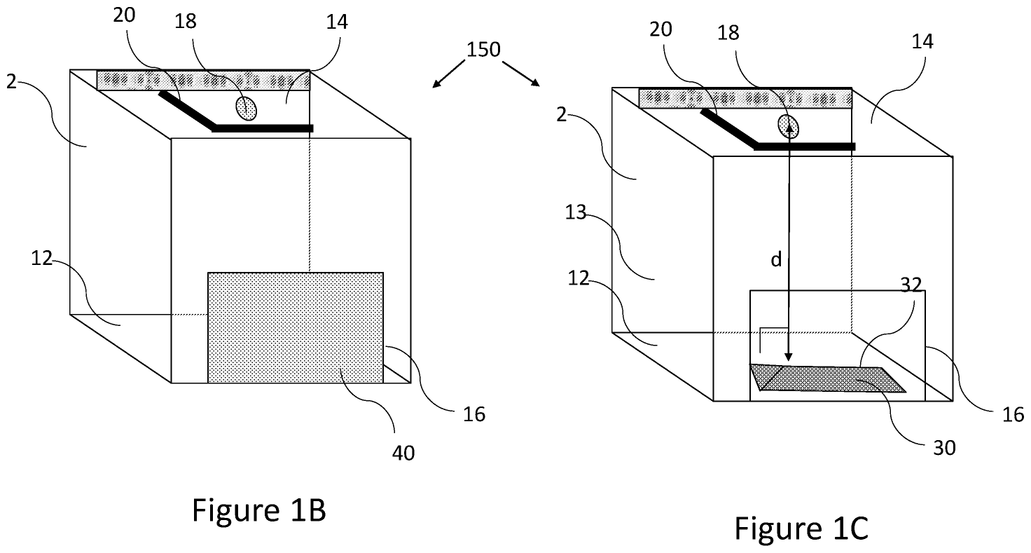

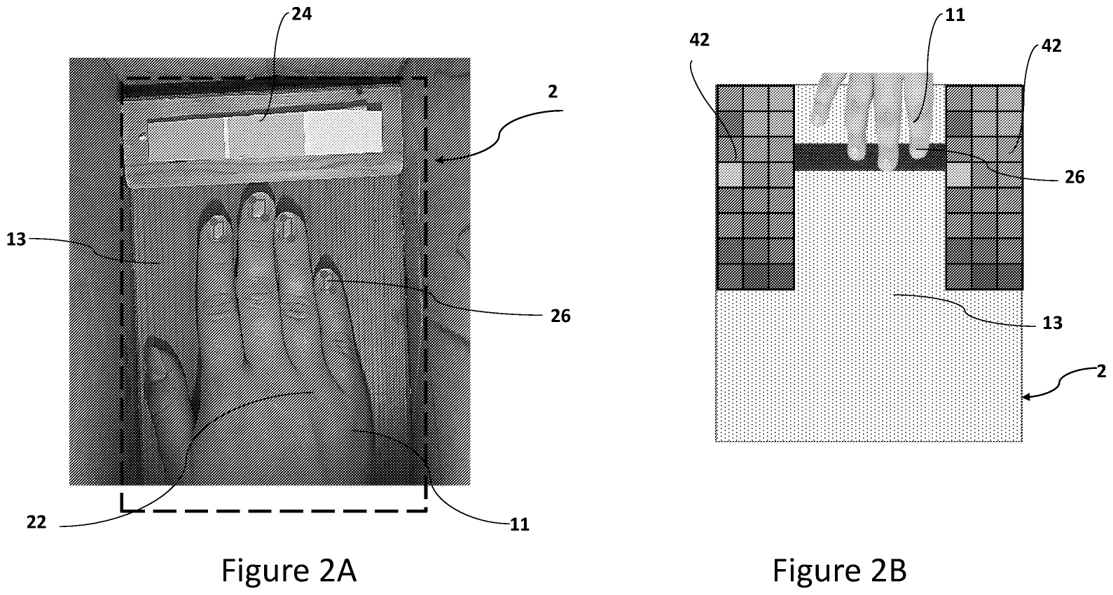

Reference is now made to FIG. 1A demonstrating a non-limiting configuration of an apparatus 100, wherein the apparatus includes a box 2. The box 2 may have four side vertical walls 10, a bottom face 12 and a top/upper face 14. In some embodiments, box 2 may have circular walls or any other shape of vertical walls. Box 2 may include un upper opening 18 adopted to allow a camera, (e.g., with a flash device) placed on top of box 2, to capture images of a subject's hand placed inside the interior space (e.g., interior space 13 illustrated in FIGS. 1C, 2A and 2B), wherein a vertical distance ‘d’ between the upper opening and the bottom of the box (illustrated in FIG. 1C) is between 5 to 40 mm.

In some embodiments, apparatus includes a side opening 16 adapted and suitable for insertion of at least the fingers of a subject, such as that the field of view of the camera includes the nail portion of the user. Optionally, side opening 16 comprises a cover made of an elastic material, in a way that substantially limits the amount of exterior light inserted within the interior space of the apparatus upon applying the subject's hand within said side opening 16.

The opening can be located at any location along the wall as long as the field of view of the camera includes the nail portion of the user.

The dimensions of side opening 16 is sufficient for insertion a subject's hand or at least fingers, while the fingers are disposed in a spread-out position (e.g., as depicted under FIG. 2). Positioning the fingers in a spread-out position may improve the hemoglobin measurements, by preventing disruption of the blood flow in case of clenched fingers.

The apparatus includes an adaptor 20 for mounting a camera (e.g., with a flash device). Optionally, the adaptor 20 is a groove suitable for mounting a smartphone. The adaptor 20 positions the camera in a position, such that a field of view of said camera captures the interior space of the box, and in a way that the camera substantially limits the amount of exterior light inserted from the adaptor to the interior space.

Top face 14 may include the adaptor 20. Alternatively, the adapter can be located at one of the side walls 10. In some embodiments, adaptor 20 mount said camera such that a field of view of the camera captures at least a portion of the subject's hands comprising the nail and the skin.

Reference is now made to FIGS. 1B and 1C demonstrating perspective views depicting another non-limiting exemplary configuration of an apparatus 150 for determining hemoglobin levels, with and without a cover, respectively, according to some embodiments of the invention. Apparatus 150 may include box 2, which is substantially the same as box 2 of apparatus 100. Box 2 may include un upper opening 18 adopted to allow a camera, (e.g., with a flash device) placed on top of box 2, to capture images of a subject's hand placed inside interior space 13, wherein a vertical distance ‘d’ between the upper opening 18 and the bottom 12 of the box (illustrated in FIG. 1C) is between 50 to 400 mm. Box 2 may further include the a side opening 16 that is configured for an insertion of said subject's hand.

In some embodiments, apparatus 150 includes the adaptor 20, attached to upper face 14 to the box, for mounting the camera and ensuring a location of the camera lens above upper opening 18. Adaptor 20 may include any device, element or component that may allow to mount a camera, such as the camera of a smartphone, to upper face 14 of the box. In a nonlimiting example, adopter 20 may allow mounting a smartphone to box 2 in only one possible way, as to avoid misaligning of the camera. Adaptor 20 may include adhesive surface, bordering elements (as illustrated), clips, or any other element that allow to detachably connect a camera (e.g., included in a smartphone) to upper face 14. In some embodiments, adaptor 20 mounts said camera such that a field of view of the camera captures the nail and the skin.

In some embodiments, apparatus 150 includes a placement pad 30 located at a bottom 12 of box 2, having a texture configured to direct said subject to place a hand portion comprising the nail and the skin in a direction facing a lens of said camera. In some embodiments, pad 30 may have a texture shaped to hold human fingers, or any other suitable shape. In some embodiments, face 32 of pad 30 may include a touch sensor (e.g., a touch screen) configured to detect placement of at least one finger/hand portion on pad 30. In some embodiments, the touch sensor may be configured to provide the location of the at least one finger/hand portion on the ped. In some embodiments, the touch sensor may send the location to a user device (e.g., the smartphone) and an application running on the user device may present instructions to the user to correct the placing of the at least one finger/hand portion on pad 30.

In some embodiments, apparatus 150 includes a cover 40 (illustrated in FIG. 1B) connected to box 2, for covering side opening 16 for substantially blocking an amount of exterior light to be inserted within said interior space 13 upon applying the camera on the adaptor and the insertion of the subject's hand via said opening. In some embodiments, the maximum amount of exterior light to be captured by the camera is at most 1 Luman. In some embodiments, cover 40 may be made from any flexible opaque material, such as, opaque fabrics, opaque polymeric sheet and the like. In some embodiments, cover 40 may be connected/connectable to box 2 in a way that allows a user to enter at least a portion of its/her hand into side opening 16.

In some embodiments, apparatus 150 includes a color scale bar 24 or 42 (illustrated in FIGS. 2A and 2B) located at said bottom of the box, displaying at least 3 colors characterized by a reflection of wavelength in the range of (i) 670-700 nm; (ii) 520-560 nm; and (iii) 450-490 nm, wherein a field of view of said camera also captures the color scale bar.

Reference is now made to FIGS. 2A and 2B, depicting the inner portions of the apparatuses. Apparatus 100 or 150 may further comprise at least one color scale bar 24 or 42, positioned in the field of view of the camera such as to assist in an image segmentation process of the nail portion of the image captured by the camera.

In some embodiments the color scale bar 24 displays at least 3 colors characterized by a reflection of wavelength in the range of (i) 670-700 nm; (ii) 520-560 nm; and (iii) 450-490 nm. In some embodiments, the color scale bar 42 displays more than 3 colors.

In some embodiments, said color scale bars 24 or 42, further displays at least one grayscale portion. According to some embodiments, said color scale bar further displays three different grayscale portions.

As discussed herein, the apparatus is used for taking an image of the nails of a subject with a camera, and determining hemoglobin levels by analyzing the color of the nails 26. Using nails as for determining hemoglobin levels is ideal since nails contain minimal amounts of melanin compared to other parts of the skin and, therefore, the primary source of color in the nails is hemoglobin. In addition, nails have low person-to-person size and shape variability. The invention is capable of providing highly accurate determination of hemoglobin levels by virtue of using the apparatus and method described herein.

The color scale bar 24 or 42 may be used for an image segmentation process of the nail portion from the image captured by the camera (e.g., differentiating the nail portion relevant for hemoglobin measurements from other portions of the image including skin and background. Advantageously, the color scale bar improves the accuracy of the measurement of hemoglobin levels.

The method determines hemoglobin levels with an accuracy of ±9 g/dL and a sensitivity of 95%.

In some embodiments, the distance between the camera located in the adaptor, and the subject's hand 11 is substantially constant. Further, the apparatus (e.g., via the opening) may provide suitable positioning for a hand of a subject in a manner that improves the focus of the images taken by the camera and reduce the error of the measurements.

In some embodiments, limiting the exterior light within the interior space 13 of the box 2 (to less than 1 Luman), and using only the flash device of the camera, improves the accuracy of the measurement of hemoglobin levels.

The apparatus, kit and method of the invention are suitable for use with a smartphone for taking the images, so that anyone with a smartphone can immediately determine the hemoglobin level anywhere and anytime.

### Kit

In some embodiments, there is provided a kit comprising
i. a box, such as box 2 defining an interior space comprising:
(a) un upper opening, such as upper opening 18, adopted to allow a camera placed on top of the box to capture images of a subject's hand placed inside the interior space, wherein a vertical distance between the upper opening and the bottom of the box is between 50 to 400 mm; and
(b) a side opening, such as side opening 18, configured for insertion of at least a subject's hand.

In some embodiments, the kits further includes,
ii. an adaptor, such as adaptor 20, attachable to an upper face of the box, for holding the camera and ensuing a location of the camera lens above the upper opening; and
iii. a placement pad, such as placement pad 30, to be located at a bottom 12 of box 2, having a texture configured to direct said subject to place a hand portion comprising a nail and skin in a direction facing a lens of said camera.

In some embodiments, the adopter is configured to mount the camera such that a field of view of said camera captures the nail and the skin.

In some embodiments, the kit further include at least one color scale bar, such as color scale bars 24 or 42, adopted to be located at said bottom of the box, displaying at least 3 colors characterized by a reflection of wavelength in the range of (i) 670-700 nm; (ii) 520-560 nm; and (iii) 450-490 nm, wherein a field of view of said camera also captures the color scale bar.

In some embodiments, the kit further includes a cover, such as cover 40, connectable to the box, and adopted to a cover the side opening for substantially blocking an amount of exterior light to be inserted within said interior space upon applying the camera on the adaptor and the subject's hand within said opening.

The box may be of any size or shape as long as it allows suitable conditions for capturing the image of the hand. In some embodiments, the apparatus can be a package of an infant milk powder.

### Method

The present invention further provides a system, a method, and/or a computer program product.

Optionally, the computer program product comprises a computer-readable storage medium. The computer-readable storage medium may have program code embodied therewith. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), via a satellite internet connection or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention In some embodiments remote connection is via radio waves (e.g. in the microwave range).

Aspects of the present invention are described herein with reference to drawings and/or diagrams of methods, apparatus (systems), and computer program products according to embodiments of the invention. It will be understood that each illustration and/or drawing, and combinations thereof, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the drawings. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the drawings.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the drawings.

In some embodiments, the program code is excusable by a hardware processor.

In some embodiments, the hardware processor is a part of the control unit.

In some embodiments, the method provided herein is for determining hemoglobin levels in a subject in need thereof using the apparatus described herein.

In some embodiments, the method receives an image from said camera captured under a defined light condition using the apparatus described herein, the image comprising a plurality of nails and skin of the subject and the color scale bar.

In some embodiments, the method operates to execute program instructions and may be further configured to segment the images of the subject's hand using at least one neural network model.

In some embodiments, the method operates to execute program instructions and may be further configured to segment the images of the subject's hand using at least one neural network model.

In some embodiments, the method operates to execute program instructions and may be further configured to transform the image data into hemoglobin levels, such as by trained neural network.

The term “image” may refer to a graphical digital image depicting a graphical representation of subject matter. In some embodiments, an image uses pixels or vector-based graphics to represent a depiction of an object, such as one or more nails. In some embodiments, an image is a stand-alone image, such as a scanned document. Additionally, or alternatively, an image is included in a collection of images, such as a frame within a video stream that includes a set of video frames.

The term “segmentation” refers to analysis of an image to determine related areas of the image. In some embodiments, segmentation is based on semantic content of the image. In some embodiments, segmentation analysis performed on an image indicates a region of the image depicting a nail area together with the color scale bar. In some embodiments, segmentation analysis produces segmentation data, such as a segmentation mask identifying the area of an image corresponding to a target object. The segmentation data indicates one or more segmented regions of the analyzed image, for example, indicating whether a given pixel in the image is part of an image region depicting a nail. Additionally, or alternatively, the segmentation data includes numerical data, such as data indicating a probability that a given pixel is an image region depicting a nail.

The term “mask” may refer to a region of interest represented by non-zero pixel values in an image. In some embodiments, a mask, object mask, or segmentation mask may refer to an image where the intensity values for pixels in a region of interest are non-zero, while the intensity values for pixels in other regions of the image are set to the background value (e.g., zero).

The term “neural network” may refer to one or more computer-implemented networks capable of being trained to achieve a goal. Unless otherwise indicated, references herein to a neural network include one neural network or multiple interrelated neural networks that are trained together. In some embodiments, a neural network (or a component of a neural network) produces output data, such as segmentation data, data indicating image features, or other suitable types of data. Examples of neural networks include, without limitation, convolutional neural networks (CNNs), recurrent neural networks (RNNs), fully-connected neural networks, encoder neural networks (e.g., “encoders”), decoder neural networks (e.g., “decoders”), dense-connection neural networks, and other types of neural networks.

Image segmentation may be used to divide a digital image into a plurality of segments. In some embodiment, an image may be segmented into objects with specific boundaries (lines, curves, etc.) or divided into elements in the image foreground or background. In particular, each pixel of an image may be marked such that pixels with the same marking, share certain characteristics. Once segmented, the image may be manipulated according to the segments, optionally by extracting the segments or blurring portions of the image.

In some embodiment, neural networks have the ability to perform the image segmentation. In some embodiments, a neural network may be trained to receive an image and in response to receipt of the image, output a segmentation mask. In some embodiments, the program creates training and testing set by masking, and provides an option to define the region of interest which indicates the nail portion. In some embodiments, the definition of region of interest is performed manually.

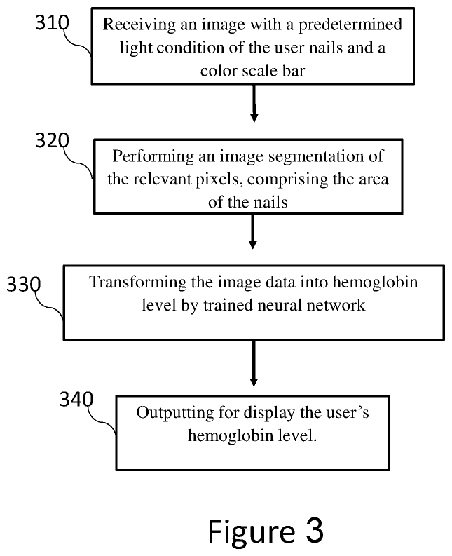

In some embodiments, the program has a series of operational steps to be performed, as can be seen in FIG. 3, the flowchart illustrates the steps for performing the specified functions and program instruction:
(i) receiving an image captured under a predetermined and defined light condition, the image comprising the fingernails of a subject and a color scale bar (step 310);
(ii) performing image segmentation of the relevant pixels relevant to the nail portions within the image (step 320);
(iii) transforming the image data into hemoglobin level by trained neural network (step 330); and
(iv) outputting for display the determined hemoglobin level of the subject (step 340).

Regions of interest indicating the nail portion may be manually selected or may be automatically selected.

In some embodiments, the received image, in step 310, are captured by a camera placed on devices 100 and/or 150 such that the lens of the camera is facing upper opening 18 and directed towards at least one hand portion comprising at least nail, placed inside box 2. The at least one hand portion may be placed on angular pad 30 as to align the at least one hand portion comprising at least nail to be directed towards the lens in optimal angle.

The regions of interest may be selected from at least one finger, at least two fingers, at least three fingers, or at least four fingers. The regions of interest may be provided using a single image, or by several images, each providing a separate region of interest. Color data is than extracted from each region and may be averaged together across fingers for each subject.

Another aspect of the invention is directed to a method of non-invasive rapid blood hemoglobin levels measurement with a mean absolute error of less that 0.95 g/dL, the method comprising receiving an image of nails of a subject captured from a camera under a defined light condition of at most 1 Luman. In some embodiments, the images may be received from a camera placed on top of apparatus 100 and/or 150 when at least a portion of a hand of the subject comprising the nail and the skin are located inside box 2.

The method may be used to collect data under substantially identical conditions, regardless of the smartphone used, and hence increase the size of the patient image pool, facilitating the incorporation of deep machine learning techniques to further refine the Hgb measurement algorithm.

### EXPERIMENTAL RESULTS

The degree of accuracy of images taken using apparatus 100 and/or 150 when the camera lens is located at various distances from the bottom 12 of box 2 or from pad 30. Table 1, shows the dependency of the mean average error on the distance ‘d’. As clearly shown there is an optimal range from 50-400 mm at which the error approximately 1±0.1.

TABLE 1

| | Mean |
|---|---|
| | Average |
| Distance | Error |
| (mm) | (±) |
| 1 | 1.62 |
| 50 | 1.03 |
| 100 | 0.96 |
| 200 | 0.94 |
| 300 | 0.99 |
| 400 | 1.11 |
| 500 | 1.26 |

The correlation between usable pixels in the image and the horizontal deviation of the location of the hand portion on pad 30 with respect to the location of the lens of the camera. The horizontal deviation is measured from the location from a theoretical vertical line extending from the camera lens to bottom surface to the location of the nail. Table 2 summarizes the percentage of usable pixels as function of bidirectional (±) horizontal deviation in mm. Usable pixels are pixels that can be used for determining hemoglobin levels.

TABLE 2

| Horizontal | |
|---|---|
| deviation | |
| of Fingernails | |
| from Directly | |
| Underneath | % |
| Camera | Useable |
| (mm) | Pixels |
| −50 | 42 |
| −30 | 63 |
| −10 | 84 |
| 0 | 91 |
| 10 | 84 |
| 30 | 65 |
| 50 | 41 |

As shown in table 2 there is great importance for the placement of users fingers and nails in box 2. Pad 30 must ensure minimal (e.g., less than ±10 mm) horizontal deviation.

In some embodiments, the distance of the camera lens from the hand portion and the horizontal deviation are important parameters of apparatuses and kits according to embodiments of the invention. Therefore, such apparatus includes a pad, such as, pad 30 to ensure minimal horizontal deviation and an opening for the camera lens located at the upper face of the box at a distance of 50 to 400 mm form the bottom of the box (e.g., the location pf pad 30).

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “substantially” refers to at least 80%, at least 85%, at least 90%, at least 92%, at least 95%, at least 96%, at least 97%, at least 98%, at least 99%, including any range or value therebetween. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1. An apparatus for determining hemoglobin levels in a subject, said apparatus comprising:
(i) a box defining an interior space comprising:
(a) un upper opening adopted to allow a camera placed on top of the box, to capture images of a subject's hand placed inside the interior space, wherein a vertical distance between the upper opening and the bottom of the box is between 50 to 400 mm; and
(b) a side opening configured for an insertion of said subject's hand;
(ii) an adaptor, attached to an upper face to the box, for holding the camera and ensuing a location of a lens of said camera above the upper opening;
(iii) a placement pad located at a bottom of the box, having a texture configured to direct said subject to place a hand portion comprising a nail and a skin in a direction facing the lens; and
wherein a field of view of said camera captures the nail and the skin.

2. The apparatus of claim 1, further comprising a color scale bar, located at said bottom of the box, displaying at least 3 colors characterized by a reflection of wavelength in the range of (i) 670-700 nm; (ii) 520-560 nm; and (iii) 450-490 nm, wherein a field of view of said camera also captures the color scale bar.

3. The apparatus of claim 1, further comprising a cover connected to the box, for covering the side opening for substantially blocking an amount of exterior light to be inserted within said interior space upon applying the camera on the adaptor and the subject's hand within said opening.

4. The apparatus of claim 3, wherein the maximum amount of exterior light to be captured by the camera is at most 1 Luman.

5. The apparatus of claim 1, wherein said camera is included in a smartphone.

6. The apparatus of claim 1, wherein said interior space has dimensions sufficient for an insertion of the hand of the subject at a spread-out position.

7. The apparatus of claim 2, wherein said color scale bar further displays at least one grayscale portion.

8. A kit comprising:
i. a box defining an interior space comprising:
(a) un upper opening adopted to allow a camera placed on top of the box to capture images of a subject's hand placed inside the interior space, wherein a vertical distance between the upper opening and the bottom of the box is between 50 to 400 mm; and
(b) a side opening configured for insertion of at least a subject's hand;
ii. an adaptor, attachable to an upper face of the box, for holding the camera and ensuing a location of the camera lens above the upper opening; and
iii. a placement pad to be located at a bottom of the box, having a texture configured to direct said subject to place a hand portion comprising a nail and skin in a direction facing a lens of said camera;
wherein a field of view of said camera captures the nail and the skin.

9. The kit of claim 8, further comprising a color scale bar, adopted to be located at said bottom of the box, displaying at least 3 colors characterized by a reflection of wavelength in the range of (i) 670-700 nm; (ii) 520-560 nm; and (iii) 450-490 nm, wherein a field of view of said camera also captures the color scale bar.

10. The kit of claim 8, further comprising a cover, connectable to the box, and adopted to a cover the side opening for substantially blocking an amount of exterior light to be inserted within said interior space upon applying the camera on the adaptor and the subject's hand within said opening.

11. A method for determining hemoglobin levels in a subject in need thereof using an apparatus comprising:
(i) a box defining an interior space comprising:
(a) un upper opening adopted to allow a camera placed on top of the box, to capture images of a subject's hand placed inside the interior space, wherein a vertical distance between the upper opening and the bottom of the box is between 50 to 400 mm; and
(b) a side opening configured for an insertion of said subject's hand;
(ii) an adaptor, attached to an upper face to the box, for holding the camera and ensuing a location of a lens of said camera above the upper opening;
(iii) a placement pad located at a bottom of the box, having a texture configured to direct said subject to place a hand portion comprising a nail and a skin in a direction facing the lens; wherein a field of view of said camera captures the nail and the skin,
and wherein the method operates to execute program instructions, and comprises the following steps:
i. receiving an image from said camera captured under a defined light condition, the image comprising a plurality of nails and skin of the subject and the color scale bar;
ii. performing an image segmentation of the pixels of the nail portions of the image;
iii. transforming the image data into hemoglobin levels by trained neural network; and
iv. outputting for display the hemoglobin level of the subject.

12. The method of claim 11, wherein said program instructions further receive as input a video from the camera.

13. The method of claim 11, wherein said program instructions further analyze a heart rate of the subject from said video.

14. The method claim 11, wherein said program instructions further receive as input one or more parameters of the subject selected from of the group consisting of: age, gender, and heart rate.

15. A method of non-invasive rapid blood hemoglobin levels measurement with a mean absolute error of less than 0.95 g/dL, the method comprising receiving an image of nails of a subject captured from a camera under a defined light condition of at most 1 Luman.

16. (canceled)
